(19) United States
(12) Patent Application Publication
(10) Pub. No.: US 2023/0001169 A1
(43) Pub. Date: Jan. 5, 2023

(54) Electrode Arrays For Electroporation, and Related Systems and Methods

(71) Applicant: Inovio Pharmaceuticals, Inc., Plymouth Meeting, PA (US)

(72) Inventors: Jay McCoy, Temecula, CA (US); Paul Fisher, SR., Fallbrook, CA (US)

(21) Appl. No.: 17/855,415

(22) Filed: Jun. 30, 2022

Related U.S. Application Data
(60) Provisional application No. 63/217,083, filed on Jun. 30, 2021.

(51) Int. Cl. A61M 37/00; A61N 1/05; A61N 1/32

(52) U.S. Cl. CPC A61M 37/00; A61N 1/0502; A61N 1/327; A61M 2037/0007

(57) ABSTRACT

An electrode array for use with an electroporation device includes a support member having a top surface and a bottom surface and defines a plurality of injection channels extending from the top surface to the bottom surface. A plurality of needle electrodes are coupled to the support member, such that distal ends of the plurality of needle electrodes extend to a needle depth below the bottom surface. The plurality of needle electrodes are arranged in a matrix pattern having rows of the needle electrodes and columns of the needle electrodes disposed along the support member. The plurality of injection channels are dispersed within the matrix pattern.

### CROSS-REFERENCE TO RELATED APPLICATIONS

The present application claims the benefit of U.S. Provisional Application No. 63/217,083, filed Jun. 30, 2021, the entire contents of which are incorporated herein by this reference.

### TECHNICAL FIELD

The present invention relates to electroporation devices, and more particularly to electrode arrays having adapted to provide increased injection volumes and a more voluminous electroporation field in tissue.

### BACKGROUND

The classical mode of administering vaccines and other pharmaceutical agents into the body tissues is by direct injection into muscle or skin tissues using a syringe and needle. Incorporating electroporative pulses of electric energy at or near the injection site is known to facilitate delivery of such vaccines or agents directly into the cells within the tissue. Such direct delivery to cells using electroporative electric pulses can have a profound clinical effect on the quality of the response of the body's metabolic and/or immune systems over that of simple syringe and needle injection. Moreover, the capability of direct delivery of agents into the cell via electroporation has enabled the effective delivery of therapeutic agents (e.g., DNA-encoded monoclonal antibodies (dMAb), expressible naked DNA encoding a polypeptide, expressible naked DNA encoding a protein, recombinant nucleic acid sequence encoding an antibody, and the like) having any number of functions, including antigenic for eliciting of immune responses, or alternatively, metabolic for affecting various biologic pathways that result in a clinical effect.

### SUMMARY

According to an embodiment of the present disclosure, an electrode array for use with an electroporation device includes a support member having a top surface and a bottom surface and defines a plurality of injection channels extending from the top surface to the bottom surface. A plurality of needle electrodes are coupled to the support member, such that distal ends of the plurality of needle electrodes extend to a needle depth below the bottom surface. The plurality of needle electrodes are arranged in a matrix pattern having rows of the needle electrodes and columns of the needle electrodes disposed along the support member. The plurality of injection channels are dispersed within the matrix pattern.

According to another embodiment of the present disclosure, an electroporation device for causing in vivo reversible electroporation in cells of tissue includes an electrode array and a plurality of injection needles. The electrode array includes a support member having a top surface and a bottom surface and defining a plurality of injection channels extending from the top surface to the bottom surface. A plurality of needle electrodes are coupled to the support member, such that distal ends of the plurality of needle electrodes extend to a needle depth below the bottom surface of the support member. The plurality of needle electrodes are arranged in a matrix pattern having rows of the needle electrodes and columns of the needle electrodes disposed along the support member. The plurality of injection channels are dispersed within the matrix pattern. The injection needles are configured to extend through at least some of the plurality of injection channels and into the tissue.

According to another embodiment of the present disclosure, an electroporation system for causing in vivo reversible electroporation in cells of tissue includes an electrode array having a support member that has a top surface and a bottom surface and defines a plurality of channels extending from the top surface to the bottom surface. A plurality of needle electrodes are coupled to the support member and extend through the plurality of channels, such that distal ends of the plurality of needle electrodes extend to a needle depth below the bottom surface of the support member. The plurality of needle electrodes are arranged in a matrix pattern having rows of the needle electrodes and columns of the needle electrodes disposed along the support member. At least some of the plurality of needle electrodes are dual-purpose needle electrodes configured to inject an agent into the tissue and to deliver one or more electroporation pulses to the tissue for causing the reversible electroporation in the cells thereof.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a side plan view of an electroporation system having a hand-held electroporation device that incorporates an electrode array, according to an embodiment of the present disclosure;

FIG. 1B is a sectional side view of a mounting portion of the hand-held electroporation device that carries the electrode array illustrated in FIG. 1A;

FIG. 1C is a top view of the electrode array illustrated in FIG. 1B, showing the electrode array having electroporation needles arranged in an exemplary 5×2 matrix and injection channels for receiving injection needles interspersed between the electroporation needles, according to an embodiment of the present disclosure;

FIG. 1D is a side view of the electroporation needle array illustrated in FIG. 1C;

FIG. 2A is a perspective view of an array having electroporation needles arranged in an exemplary 6×4 matrix and injection channels for receiving injection needles interspersed between the electroporation needles, according to an embodiment of the present disclosure;

FIG. 2B is a side view of the array illustrated in FIG. 2A;

FIG. 2C is a bottom view of the array illustrated in FIG. 2A;

FIG. 2D is a top view of the array illustrated in FIG. 2A;

FIG. 3A is a bottom view of an array similar to the array shown in FIGS. 2A-2D but having different inter-electrode spacing, according to an embodiment of the present disclosure;

FIG. 3B is a top view of the array illustrated in FIG. 3A;

FIG. 3C is a bottom view showing calculated electric field magnitudes of the array illustrated in FIG. 3A;

FIG. 4 is a bottom view of a modular array having electroporation needles arranged in an exemplary 6×4 matrix and injection channels for receiving injection needles interspersed between the electroporation needles, according to an embodiment of the present disclosure;

FIG. 5A is a perspective view of an electroporation system that includes an array having dual-purpose injection and electroporation needles arranged in a matrix, specifically in which the needle electrodes are dual-purpose injection needles that are configured to both deliver injectate to target tissue and deliver one or more electroporative pulses to the target tissue, according to an embodiment of the present disclosure;

FIG. 5B is a perspective view of an array assembly of the electroporation system illustrated in FIG. 5A;

FIG. 5C is a plan view showing the array assembly inserted within muscle tissue;

FIG. 6A is a bottom view of an electroporation array assembly having electroporation needles arranged in a 3×2 matrix and injection channels that are eccentrically offset from the electroporation needles, according to an embodiment of the present disclosure;

FIG. 6B is a side view of the electroporation array assembly illustrated in FIG. 6A;

FIG. 7A is a bottom view of an electroporation array assembly having electroporation needles arranged in a 3×2 matrix and injection channels that are in-line with the rows of electroporation needles, according to an embodiment of the present disclosure;

FIG. 7B is a side view of the electroporation array assembly illustrated in FIG. 7A;

FIGS. 8A-8C are diagram views showing example pulsing patterns for the electrode array illustrated in FIGS. 7A-7B;

FIGS. 9A-9B are plan views showing the electroporation array assembly of FIG. 7A inserted within muscle tissue at parallel (FIG. 9A) and perpendicular (FIG. 9B) orientations relative to the muscle fibers; and

FIG. 9C is a set of diagram views showing calculated electric field magnitudes of an electrode row of the array illustrated in FIGS. 7A-7B at various orientations with respect to the muscle fibers.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

The term “agent”, as used herein, means a polypeptide, a polynucleotide, a small molecule, or any combination thereof. The agent may be a recombinant nucleic acid sequence encoding an antibody, a fragment thereof, a variant thereof, or a combination thereof. The agent may be a recombinant nucleic acid sequence encoding a polypeptide or protein. The agent may be formulated in water or a buffer, such as saline-sodium citrate (SSC) or phosphate-buffered saline (PBS), by way of non-limiting examples.

The term “intradermal” as used herein, means within the layer of skin that includes the epidermis (i.e., the epidermal layer, from the stratum corneum to the stratum basale) and the dermis (i.e., the dermal layer).

The term “intramuscular” as used herein, means within muscle tissue, including skeletal muscle tissue and smooth muscle tissue.

The term “adipose”, as used herein, means the layer containing adipocytes (i.e., fat cells) that reside in the subcutaneous layer.

The term “electroporation”, as used herein, means employing an electrical field within tissue that temporarily and reversibly increases the permeability and/or porosity of the cell membranes of cells in the tissue, thereby allowing an agent, for example, to be introduced into the cells. It should be appreciated that the type of electroporation disclosed herein refers to reversible electroporation (also referred to as “reversible poration”), meaning that the electroporated cell membranes (or at least a majority thereof) return to a substantially non-permeable and/or non-porous state following electroporation.

The term “electroporation field”, as used herein, means an electric field capable of electroporating cells. In instances where an electric field includes a portion that is capable of electroporating cells and another portion that is incapable of electroporating cells, the “electroporation field” refers specifically to that portion of the electric field that is capable of electroporating cells. Thus, an electroporation field can be a subset of an electric field.

The embodiments disclosed herein pertain to electroporation devices that employ an electrode array having a plurality of needle electrodes arranged in a pattern and also having a plurality of fluid injection channels interspersed within the pattern. The array with the plurality of fluid injection channels allows greater injection volumes for increased spatial dispersion within a more voluminous electroporation field within target tissue. This can allow agent uptake into target cells on a greater scale, including within intradermal (ID) tissue, adipose tissue, and intramuscular (IM) tissue.

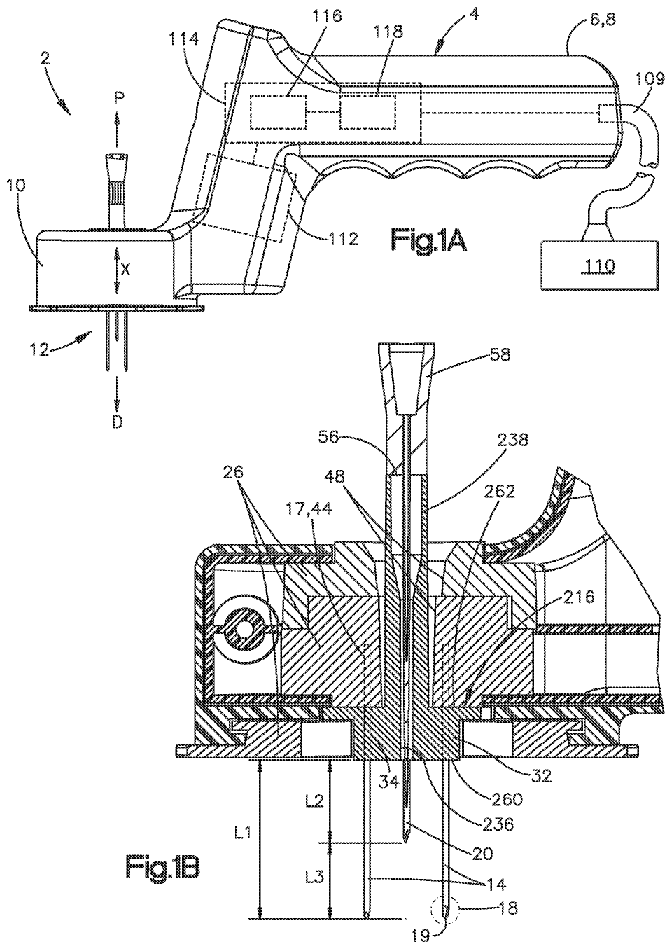

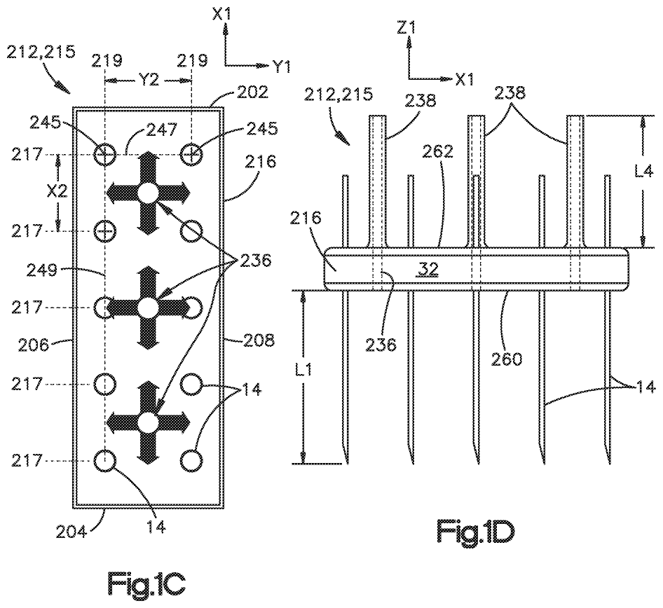

Referring to FIGS. 1A-1B, an electroporation system 2 according to an exemplary embodiment of the present disclosure includes a hand-held electroporation device 4 that includes a housing 6. The hand-held electroporation device 4 can also be referred to as an “applicator” 4. The electroporation device 4 includes a handle 8 and a mounting portion 10 (also referred to herein as a “mounting head” or “applicator head” 10) extending distally from the handle 8. The handle 8 and applicator head 10 can be defined by the housing 6. The applicator head 10 can carry an array assembly 212 that includes one or more electrodes, 14 such as a plurality of electrodes 14 in a spatial arrangement, which arrangement can be referred to as an “electrode array” 215. The electrodes 14 extend from a support member 216 in a distal direction D that is opposite a proximal direction P. The electrodes 14 of this embodiment are penetrating electrodes that have distal tips 18 configured to penetrate tissue, particularly for penetrating through dermal tissue and into muscle tissue. One or more and up to all of the distal tips 18 can be a trocar tip having planar surfaces that converge to a point at a distal end 19 of the electrode 14, by way of a non-limiting example.

The electrodes 14 are configured to deliver one or more pulses of electrical energy to cells of the target tissue, specifically for reversibly electroporating the cells. The device 4 includes circuitry for providing electrical communication between the electrodes 14 and an energy source 110. As shown, the circuitry can be configured to connect with one or more cables 109 configured to couple with an energy source 110 located remote from the hand-held electroporation device 4, such as a power generator. Additionally or alternatively, the circuitry can be configured to connect with an on-board energy source, such as a battery unit disposed within the housing 6.

The energy source 110 can be in electrical communication with a pulse generator 112, such as a waveform generator, for generating and transmitting an electric signal in the form of one or more electrical pulses having particular electrical parameters to the electrodes 14 for electroporating cells within the tissue. Such electrical parameters include electrical potential (voltage), electric current type (alternating current (AC) or direct current (DC)), electric current magnitude (amperage), pulse duration, pulse quantity (i.e., the number of pulses delivered), and time interval or “delay” between pulses (in multi-pulse deliveries). The pulse generator 112 can include a waveform logger for recording the electrical parameters of the pulse(s) delivered. The pulse generator 112 can be in electrical communication with a control unit 114 (also referred to herein as a “controller”), which can include a processor 116 configured to control operation of the electroporation system 2, including operation of the pulse generator 112. The processor 116 can be in electronic communication with computer memory 118, and can be configured to execute software and/or firmware including one or more algorithms for controlling operation of the system 2.

The processor 116 can be in electrical communication with a user interface, which can be located on the device 4 or remote from the device 4. The user interface can include a display for presenting information relating to operation of the system 2 and inputs, such as a keypad or touch-screen, that allow a physician to input information, such as commands, relating to operation of the system 2. It should be appreciated that the interface can be a computer interface, such as a table-top computer or laptop computer, or a hand-held electronic device, such as a smart-phone or the like.

The applicator head 10 is configured to receive at least one fluid delivery device that includes an elongate tubular member, which in the embodiments disclosure herein is an injection needle 20, configured to deliver an injectate to a target region of tissue. Preferably, the applicator head 10 is configured to receive a plurality of fluid delivery devices (e.g., injection needles 20), as described in more detail below.

As shown in FIG. 1B, the hand-held electroporation device 4 can include one or more mounting members 26 for mounting the support member 216 to the applicator head 10. The mounting members 26 can define respective apertures through which portions of the electrodes 14 extend. The support member 216 can include a hub or platform 32, which can define a plurality of electrode apertures 34, through which the electrodes 14 can extend, respectively. In this manner, the spacing of the electrode apertures 34 in the platform 32 can define the pattern of the electrode array 212. The support member 216 defines a plurality of injection channels 236, through which the injection needle 20 can extend. The support member 216 preferably also includes a plurality of elongated proximal tube formations 238 (also referred to herein as “chimneys” or “risers”) that extend from a top surface 262 of the platform 32. The chimneys 238 define proximal extensions of the injection channels 36 from the platform 32. The platform 32 can be configured to abut one or more of the mounting members 26 when the array assembly 212 is in an assembled configuration and coupled to the applicator head 10.

It should be appreciated that at least one the mounting members 26 can define a plurality of sockets 44 arranged correspondingly with the electrode apertures 34 of the support member 216 for receiving proximal ends 17 of the electrodes 14 and providing electrical communication between the pulse generator 112 and the electrodes 14. Additionally, one or more of the mounting members 26 can also define respective injection channels 48 that are in alignment with the injection channel 236 of the support member 216 and through which the chimneys 238 can extend.

As shown, the chimneys 238 can protrude proximally from the applicator head 10 when in the assembled configuration. A distal end 56 of the chimney 238 can be configured to mount with a connection member 58 (also referred to herein as a “connector”) attached to the injection needle 20. The connector 58 is configured to couple with a reservoir of the injectate, such as a syringe, a single-dose cartridge, an injection manifold, and the like. As shown, the connector 58 can be a Luer-type connector, although other connector types and designs are within the scope of the present embodiments.

In some embodiments, the electroporation system 2 can employ the CELLECTRA® 2000 system, which has an external, battery powered pulse generator 112 (i.e., the CELLECTRA® Pulse Generator) that is connected via cable to the hand-held electroporation device 4, which can be an adapted version of the CELLECTRA® 5P-IM Applicator, by way of non-limiting examples. It should be appreciated that the array assembly 212 is preferably a sterile disposable array assembly 212. The electrodes 14 can be constructed of stainless steel and can be gold-coated for enhanced conductivity. The injection needles 20 can be pre-packaged with the array assembly 212. It should be appreciated that the CELLECTRA® products and components described above are produced by Inovio Pharmaceuticals, Inc., headquartered in Plymouth Meeting, Pa., United States.

As shown in FIG. 1B, the array assembly 212 is preferably configured to control a maximum depth L1 at which the electrodes 14 penetrate the surface of the subject's skin. This depth L1, also referred to herein as “electrode penetration depth” or “electrode depth,” can be governed by a contact or “stop” surface 260 of the array assembly 212 that is configured to abut the subject's skin and halt further advancement of the electrodes 14 into the tissue. As shown, the stop surface 260 can be defined by a distal or bottom surface of the support member 216, by way of a non-limiting example. The array assembly 212 is preferably also configured to control a maximum depth L2 of the injection needles 20, measured from the stop surface 260 to the distal ends of the injection needles 20. This depth L2 can also be referred to herein as “injection depth.” The support member 216 is preferably configured such that the injection depth L2 is shallower than the electrode depth L1 by an injection offset distance L3, which is tailored so that the injected agent is located primarily within the electroporation field created by the electrodes 14. It should be appreciated that the depths L1, L2 can be adjusted as needed to specifically target intradermal (ID) tissue, adipose tissue, intramuscular tissue (IM), or any combination of the foregoing tissues, depending on patient needs.

Referring now to FIGS. 1C-1D, the example support member 216 can carry the needle electrodes 14 so that the electrode array 215 is a grid or “matrix” pattern. The illustrated embodiment employs a matrix having five (5) rows 217 and two (2) columns 219 of electrodes 14 (i.e., a 5×2 electrode array 215, in which each row has two electrodes, and each column has five electrodes). The rows 217 are spaced at intervals along a longitudinal direction X1, while the columns 219 are spaced at intervals along a lateral direction Y1 that is substantially perpendicular to the longitudinal direction X1. In this manner, the array 215 can be elongated along the longitudinal direction X1. It should be appreciated that the electrodes 14 of each row 217 can be aligned along a row axis 247, which can intersect central axes 245 of the electrodes 14 in the row 217. Additionally, the electrodes 14 of each column 219 can be aligned along a column axis 249, which can intersect the central axes 245 of the electrodes 14 in the row 219. The array 215 can employ equidistant row and column spacing X2, Y2, although in other embodiments the row spacing X2 can differ from the column spacing Y2. The row and column spacing X2, Y2 is preferably measured between adjacent row axes 247 and column axes 249, respectively. The electrodes 14 can be configured similarly to those described above with respect to the circular pattern electrode arrays 15, although in other embodiments the electrodes 14 of the present array 215 can be adapted as needed.

The support member 216 has first and second ends 202, 204 opposite each other along the longitudinal direction X1 and opposed first and second sides 206, 208 opposite each other along the lateral direction Y1. The bottom surface 260 of the support member can effectively define the stop surface, as mentioned above. As shown, the support member 216 can include three (3) injection channels 236, which can be aligned with each other along the longitudinal direction X1 and can be equidistantly spaced between the first and second columns 219. A first one of the injection channels 236 can also be equidistantly positioned between the first and second rows 217, a second one of the injection channels 236 can be laterally aligned in the third row 217, and a third one of the injection channels 236 can be equidistantly positioned between the fifth and sixth rows 217. Each chimney 238 can be configured to receive a respective injection needle 20, which can be configured according to any of the embodiments described above. As shown in FIG. 1C, the chimneys 238 can extend from the upper surface 262 of the support member 216 proximally to a chimney height of L4 along a vertical direction Z1, which height L4 can be configured to place the distal ends of the injection needles 20 at a favorable position relative to distal ends 19 of the electrodes 14, such as at a favorable injection offset distance L3 described above.

As shown in FIG. 1D, the injection needles 20 can each eject their injectate, which can disperse radially outward toward the adjacent needle electrodes 14. By employing multiple injection channels 236, the array 215 can be configured to disperse greater volumes of injectate within larger electroporation fields. According to one example of the present embodiment, the array 215 can be configured to deliver a total injection volume of about 3 mL from the injection needles 20, particularly at 1 mL per injection needle 20. It should be appreciated that, when used for intramuscular (IM) electroporation, the elongated array 215 allows a physician to orient the array 215 so that that the longitudinal direction X1 generally aligns with the direction of muscle fiber extension, thereby further enhancing the fluid dispersion in the muscle tissue of the patient.

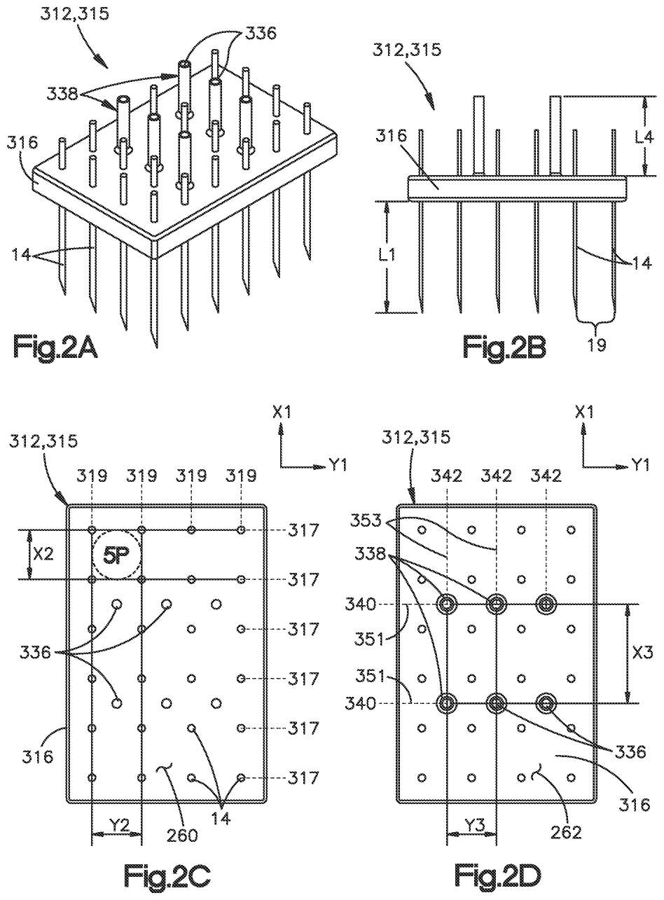

Referring now to FIGS. 2A-2D, another example array assembly 312 includes a support member 316 having an array 315 of needle electrodes 14 arranged in a matrix having six (6) rows 317 and four (4) columns 319 (i.e., a 6×4 matrix electrode array 315). As above, the rows 317 are spaced at intervals along the longitudinal direction X1, while the columns 319 are spaced at intervals along the lateral direction Y1, such that the array 315 can be elongated along the longitudinal direction X1. The array 315 can employ equidistant row and column spacing. By way of a non-limiting example, the rows 317 can be spaced from each other at a distance X2 of about 10 mm and the columns 319 can be spaced from each other at a distance Y2 of about 10 mm. It should be appreciated that such 10 mm spacing approximates the diameter of the circular electrode array of the CELLECTRA® 5P-IM Array, as shown for reference in FIG. 2C.

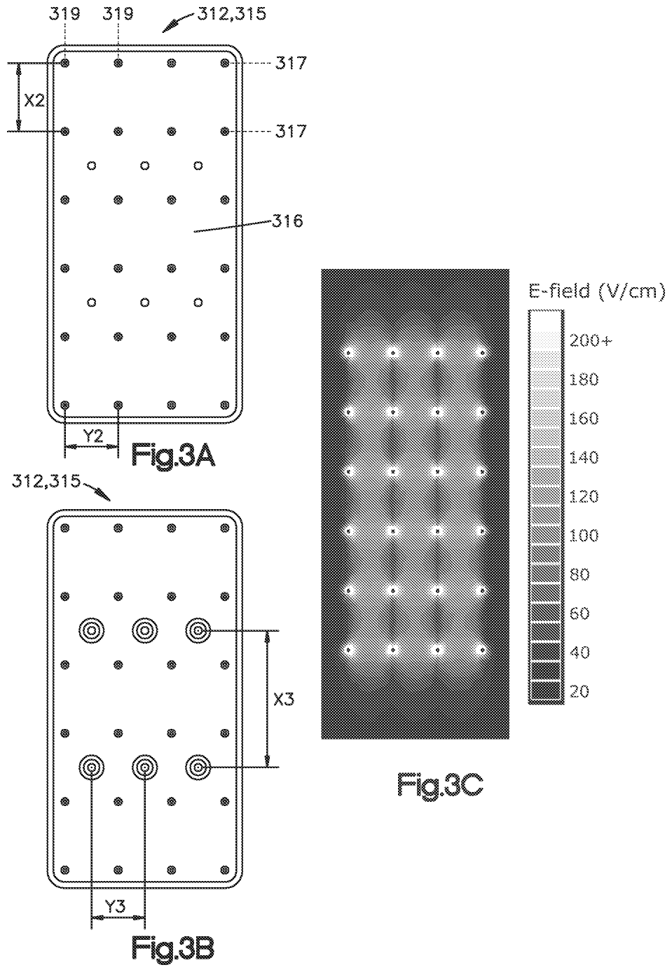

In other embodiments, as shown in FIGS. 3A-3B, the row spacing can differ from the column spacing. In this example, the columns can be spaced at distances X2 of about 10 mm, and the rows can be spaced at distances Y2 of about 7.5 mm. Additional spacing distances are discussed below.

The support members 316 of the arrays 315 shown in FIGS. 2A-2B preferably includes a plurality of injection channels 336, which can be defined within vertically elongated chimneys 338. As shown, the plurality of injection channels 336 can include six (6) injection channels 336, which can be arranged along two (2) rows 340 of channels, such as a first row 340 of channels 336 equidistantly spaced between the second and third rows 319 of electrodes 14, and a second row 340 of channels 336 equidistantly spaced between the fourth and fifth rows 319 of electrodes 14. As shown in FIG. 2D, the channel rows 340 can be spaced from each other at spacing distance X3, as measured between respective channel row axes 351 that intersect central axes 355 of the injection channels 336 in the channel row 340. In the illustrated embodiment, spacing distance X3 is 2× the electrode row spacing distance X2. The channels 336 can also be arranged into columns 342 of channels 336, such as a first, second, and third column 342 of channels 336. The channel columns 342 can be spaced from each other at spacing distance Y3, as measured between respective channel column axes 353 that intersect the central axes 355 of the injection channels 336 in the channel column 342. In the illustrated embodiment, spacing distance Y3 is equivalent to the electrode column 319 spacing distance.

According to one example of the present embodiments, the arrays 315 can be configured to deliver a total injection volume of about 6 mL from the injection needles 20, particularly at 1 mL per injection needle 20. It should be appreciated that the arrays 315 can be used for delivering injection volumes greater than 6 mL and less than 6 mL. As with the array 215 described above, the present arrays 315 can be oriented favorably with respect to the direction of muscle fiber extension, thereby enhancing the fluid dispersion in the muscle tissue. Additionally, the chimneys 338 have heights L4 that can be configured to place the infusion regions of the injection needles 20 at a favorable position relative to distal ends 19 of the electrodes 14. It should be appreciated that the electrode and channel spacing distances X2, Y2, X3, Y3, electrode depths L1, and/or the chimney heights L4 of the matrix arrays 215, 315 described above can be varied as needed. For example, spacing distances X2, Y2, X3, Y3 can be in a range from about 2.5 mm to about 50 mm, and more particularly in a range from about 4.0 mm to about 20 mm, and more particularly in a range from about 5.0 mm to about 15.0 mm. The electrode spacing distances X2, Y2 along the direction of muscle fiber extension is preferably in a range of about 10.0 mm to about 15.0 mm. The electrode spacing distances X2, Y2 along a directional that is perpendicular to the direction of muscle fiber extension is preferably in a range of about 5.0 mm to about 10.0 mm. It should be appreciated that the foregoing spacing distances can be adapted particular to the anatomy of the target tissue, particularly when the target tissue has anisotropic electrical and fluidic properties.

Referring now to FIG. 3C, a computer model illustrates an example of an electric field generated by the array 315 shown in FIGS. 3A-3B. As shown, the electric field can have a substantially even field magnitude, shown in V/cm, along the longitudinal direction X1 between adjacent columns. In this manner, the array 315 can provide both favorable longitudinal fluid dispersion, and favorable “smooth” electroporation fields along the longitudinal direction X1. The physician can take advantage of such smooth electroporation fields by orienting the array 315 in a favorable manner relative to the underlying target tissue. For example, when used for IM electroporation, the physician can orient the array 315 so that the longitudinal direction coincides with the direction of muscle fiber extension.

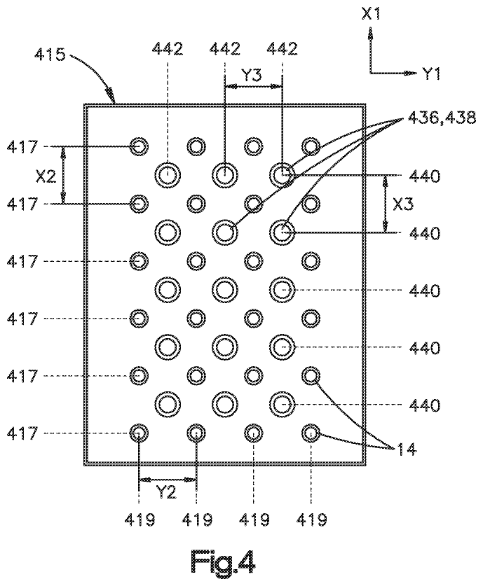

In further embodiments, the matrix arrays 215, 315 can be further configured for selective or “modular” use of the electrodes 14 and/or injection channels 236, 336 thereof. Referring now to FIG. 4A, an example array 415 having electrodes 14 arranged in a matrix, such as a 6×4 matrix with even electrode row 417 and column 419 spacing X2, Y2, by way of a non-limiting example, can include a total of fifteen (15) chimneys 438 (and channels 436), arranged in rows 440 and columns 442 in a 5×3 chimney array configured such that each chimney 438 is equidistantly spaced between the adjacent columns 419 and rows 417 of the electrodes 14. The array 415 can include circuitry for connecting each electrode 14 individually to the pulse generator 112, such that the pulse generator 112 can deliver electroporation pulses to any subset of the electrodes 14. Similarly, any subset of the chimneys 438 can be employed to receive a respective injection needle 20. In this manner, a single matrix array 438 can provide the functionality of numerous matrix arrays 438. For example, the depicted 6×4 matrix array can be selectively employed as any of a 1×1, 1×2, 1×3, 1×4, 2×1, 2×2, 2×3, 2×4, 3×1, 3×2, 3×3, 3×4, 4×1, 4×2, 4×3, 4×4, 5×1, 5×2, 5×3, 5×4, 6×1, 6×2, 6×3, and 6×4 electrode array, utilizing any one of a 1×1, 1×2, 1×3, 2×1, 2×2, 2×3, 3×1, 3×2, 3×3, 4×1, 4×2, 4×3, 5×1, 5×2, and 5×3 chimney array.

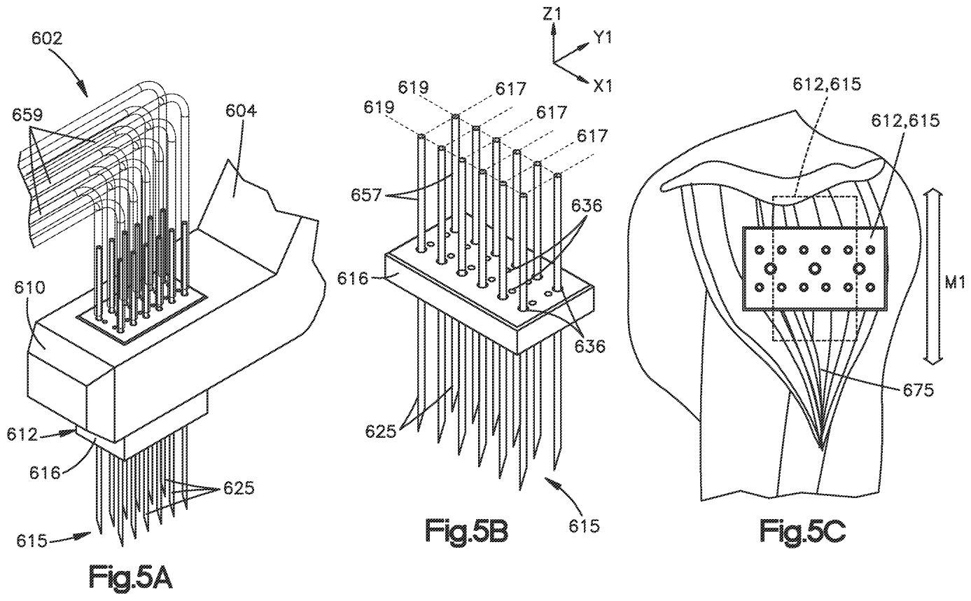

Referring now to FIGS. 5A-5C, an example of an electroporation system 602 is shown that includes an electrode array assembly 612 having a plurality of needle electrodes 625 arranged in rows 617 and columns 619 in a matrix array 615, generally similar to the embodiments described above. However, in the present embodiment, one or more and up to all of the electrodes 20 in the matrix array 615 can be a dual-purpose injection needle electrode 625 that is configured to both inject fluid within target tissue and also to deliver one or more electroporative pulses to the target tissue.

The electroporation system 602 of this embodiment can include tubing 659 for delivering the fluid injectate to each dual-purpose injection needle electrode 625 in the matrix array 615. The tubing 659 can connect proximal ends 657 of the dual-purpose injection needle electrodes 625 to a reservoir, such as via a manifold of a reservoir assembly and/or via a plurality of individual reservoirs. The array assembly 612 can be configured to couple with an applicator head 610 of a hand-held electroporation device 604. For example, the array assembly 612 can include a support member 616 configured to couple with one or more complimentary mounting members of the applicator head 610, similar to the manner described above with reference to FIG. 1B. The dual-purpose electrodes 625 can extend through dual-purpose channels 636 defined through the support member 616. It should be appreciated that the support member 616 can be employed in modular fashion, similar to the manner described above with reference to FIG. 4. For example, the dual-purpose electrodes 625 can be inserted within a select sub-set of the available dual-purpose channels 636, which sub-set can be selected based on the fluid delivery and electroporation field parameters needed, which parameters (and thus sub-set selection) can be adapted to the target tissue. It should be appreciated that the matrix array 615 can employ various combinations and patterns of needle electrodes 14, injection needles 20, and dual-purpose injection needle electrodes 625.

As shown in FIG. 5C, the matrix array 615 can be placed with respect to muscle tissue 675 so that the dual-purpose injection needle electrodes 625 are oriented as desired with respect to the muscle tissue, particularly with respect to the direction of muscle fiber extension M1. For example, the matrix array 615 can be oriented so that the longitudinal direction X1 of the array 615 extends along the direction of muscle fiber extension M1, as indicated by the array 615 position shown in dashed lines. Alternatively, the physician can elect to orient the array 615 so the longitudinal direction X1 is oriented substantially perpendicular to the direction of muscle fiber extension M1, which can therefore provide for the fluid injection to be distributed along a greater number of individual muscle fiber striations. Such selective orientations and usages of the array 615 can be further tailored by the application of the pulsing pattern with respect to specific sub-sets of dual-purpose electrodes, which pulsing patterns can be adapted to focus the EP field along the direction of muscle fiber extension M1. These is configurations and usages can also take advantage of the fact that, during EP electrical current flow, the impedance is reduced when directed in the same direction as the muscle fibers. Moreover, the direction of fluid ejection from the injection needles 20, 625 can also be expected to experience less mechanical impedance to fluid flow, which can allow for beneficial drug distribution along the electroporation field.

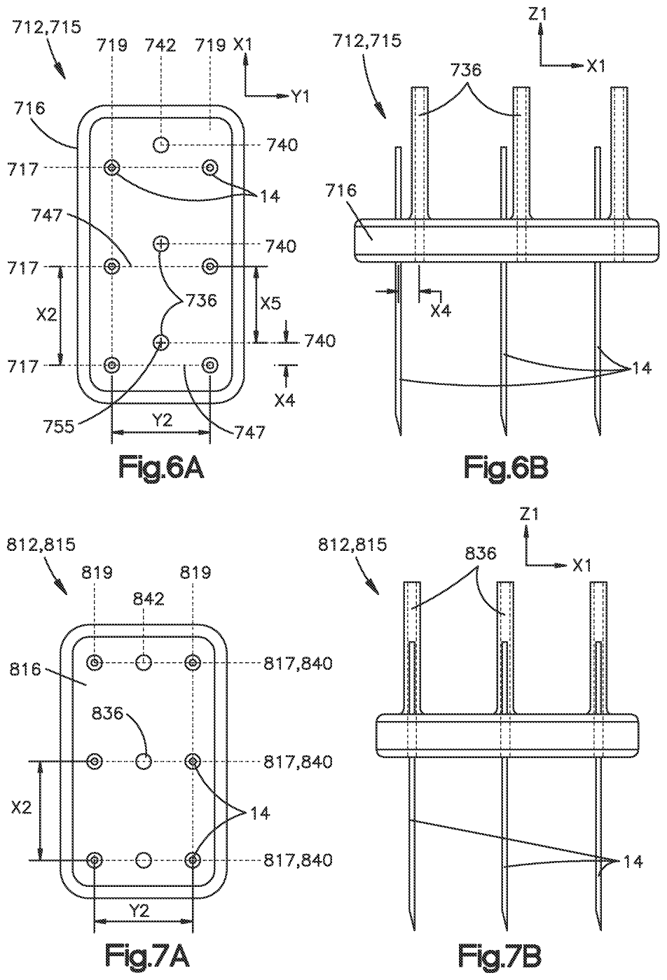

Referring now to FIGS. 6A-6B, an example embodiment of an array assembly 712 is shown having a matrix electrode array 715 coupled to a support member 716. In this example embodiment, the matrix array 715 includes a plurality of needle electrodes 14 arranged in rows 717 and columns 719 and having injection channels 736 located between the needle electrodes 14, generally similar to the embodiments described above with reference to FIGS. 1C-3B and 4-5C. However, in the present embodiment, one or more and up to all of the injection channels 736 is eccentrically offset from adjacent rows 717 and/or adjacent columns 719. As used herein with respect to an injection channel 736 and an adjacent row 717 and/or adjacent column 719, the phrase “eccentrically offset” means that the injection channel 736 is spaced from the nearest row 717 and/or column 719 along a respective direction and at a respective offset distance that is less than a distance along the respective direction between the injection channel 736 and the next nearest row 717 and/or column 719.

In the illustrated embodiment, each of the injection channels 736 is eccentrically offset from the respective nearest row 717 along the longitudinal direction X1. In particular, each injection channel 736 of the illustrated embodiment is longitudinally spaced from the nearest row 717 at an offset distance X4 that is less than a secondary offset distance X5 between the injection channel 736 and the next nearest row 717. The offset distance X4 and the secondary offset distance X5 are measured between the central axis 755 of the injection channel 736 and the nearest electrode row axis 747 and the next nearest electrode row axis 747, respectively. The offset distance X4 can be quantified as a factor (i.e., multiple) of the secondary offset distance X5. For example, the offset distance X4 can range from a factor of about 0.001 to a factor of about 0.999 of the secondary offset distance X5.

According to a non-limiting example of the illustrated embodiment, the matrix array 715 has six (6) electrodes 14 arranged in a 3×2 matrix (i.e., three (3) rows 717 and two (2) columns 719), with equidistant row and column spacing X2, Y2. The injection channels 736 are arranged in a 3×1 channel array (i.e., three (3) rows 740 and one column 742 of channels 136) such that each injection channel 736 is eccentrically offset from the nearest row 717 of electrodes 14 at equidistance offset distances X4. In this example, each offset distance X4 is a factor of about 0.25 of the respective secondary offset distance X5. In particular, in this example the electrode row spacing X2, electrode column spacing Y2, and the channel row spacing X3 are each about 10 mm, with the injection channels eccentrically offset at an offset distance X4 of about 2.5 mm along the longitudinal direction X1. It should be appreciated that any of these spacing distances X2, Y2, and offsets X4, X5 can be adjusted as needed.

It should also be appreciated that, in other embodiments, the injection channels 736 can be eccentrically offset from one of the electrode columns 719 along the lateral direction Y1. It should yet also be appreciated that the number of electrodes 14 and/or injection channels 736 in the matrix array 715 can be reduced or increased as needed based on various factors, such as the target treatment location, target tissue, and injection volume, by way of non-limiting examples. For example, the matrix array 715 can be increased to include one or more additional rows 717 and/or columns 719 of electrodes 14 and/or one or more additional rows 740 and/or columns 742 of injection channels 736, such that the injection channels 736 are eccentrically offset from the electrode rows 717. It should further be appreciated that the matrix array 715 can employ a combination of eccentrically offset injection channels 717 and injection channels 717 that are not eccentrically offset (such as by being located equidistantly between respective electrodes 14 or by being aligned with a respective electrode row 717). The matrix array 715 of the present embodiment provides significant advantages for electroporation treatment. One such advantage is that by employing multiple injection channels 736 within the electrode array 715, the agent dosage can be fractionated among multiple injection sites. This is expected to enhance fluid dispersion in target tissue.

Referring now to FIGS. 7A-7B, in another example embodiment, an array assembly 812 has a support member 816 that includes a matrix array 815 configured similar to the embodiment described above with reference to FIGS. 6A-6B. As with the aforementioned embodiment, the matrix array 815 has six (6) electrodes 14 arranged in a 3×2 matrix, with equidistant electrode row and column spacing X2, Y2, and three (3) injection channels 836 arranged in a 3×1 channel array. In the present embodiment, however, the injection channels 836 are aligned with the rows 817 of electrodes 14, such that the injection channels 836 are intersected by the respective electrode row axes 847. In one non-limiting example of the matrix array 815, the array 815 can employ an electrode row spacing X2, electrode column spacing Y2, and channel row spacing X3 that are each about 10 mm. It should be appreciated that any of these spacing distances X2, Y2, X3 can be adjusted as needed.

The matrix array 815 of the present embodiment provides significant advantages for electroporation treatment. As with the matrix arrays described above, the array 815 employs multiple injection channels 836 that allows fractionating the agent dosage among multiple injection sites. Moreover, the dispersed injectate at the multiple injection sites can be targeted with respective electroporation fields delivered by respective subsets of electrodes 14 in the array 815. Another advantage is that the matrix array 815 can employ a pulse pattern that enhances co-localization of the electroporation fields with the delivered fluid dispersions from the injection channels 836 aligned with the electrode rows 817. In particular, the matrix array 815 can employ a pulse pattern that delivers pulses between electrode pairs in each row 817, thereby directing the pulses across the area underneath the injection channels 836. This better co-localizes the electroporation fields with the fluid dispersions emanating from injection needles 14 extending through the injection channels 836, as described in more detail below.

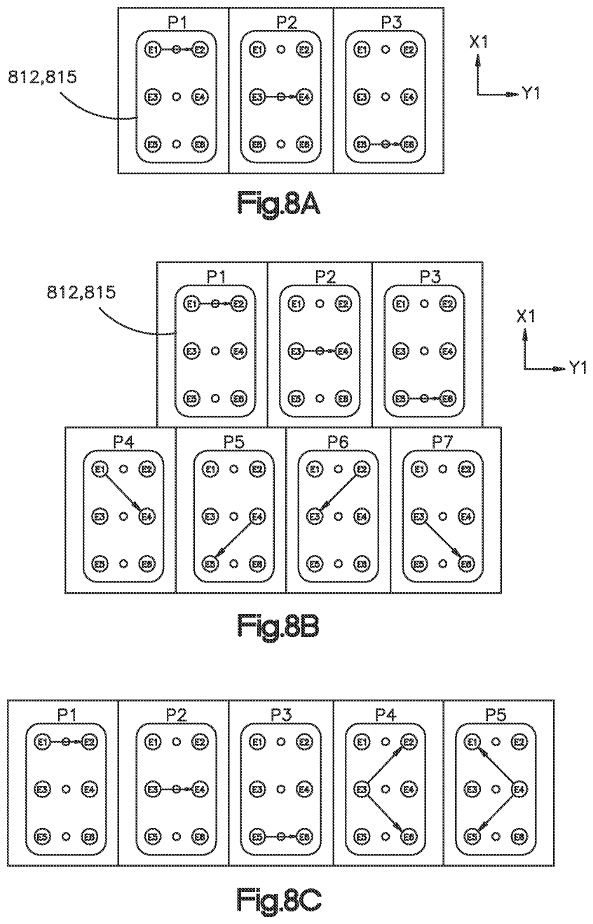

Referring now to FIG. 8A, an example pulse pattern will be described for the matrix array 81 shown in FIGS. 7A-7B. For purposes of illustrating the pulse pattern, the electrodes 14 of the matrix array 815 will be referred to by electrode positions E1-E6, in which electrode positions E1 and E2 are on a first electrode row 817, electrode positions E3 and E4 are on a second electrode row 817, and electrode positions E5 and E6 are on a third electrode row 817. In this example, the pulse pattern includes three (3) pulses, of which the first pulse P1 is delivered between E1 and E2, the second pulse P2 is delivered between E3 and E4, and the third pulse P3 is delivered between E5 and E6. In another example, the pulse pattern shown in FIG. 8A can be repeated, providing a pulse pattern having two identical pulse trains and a total of six (6) pulses. Such a repeated pulse pattern provides two pulses per electrode pair, which can facilitate enhanced electroporation results.

Referring now to FIG. 8B, in an additional example, a pulse pattern can employ the three pulses P1-P3 shown in FIG. 8A, plus four (4) additional pulses P4-P7 delivered diagonally between adjacent electrode rows 817 and columns 819. In this particular example, the fourth pulse P4 is delivered between E1 and E4, the fifth pulse P5 is delivered between E4 and E5, the sixth pulse P6 is delivered between E2 and E3, and the seventh pulse P7 is delivered between E3 and E6. The four (4) diagonal pulses P4-P7 can be beneficial for co-localizing the electroporation fields with any injectate that dispersed between the electrode rows 817 along the longitudinal direction X1.

Referring now to FIG. 8C, in a further example for co-localizing the electroporation fields with injectate that dispersed longitudinally between the electrode rows 817, a pulse pattern can effectively replace pulses P4-P7 shown in FIG. 8B with two (2) alternative pulses P4-P5 that each split the current diagonally from the center row 817 to the first and third rows 817. In particular, in this example the fourth pulse P5 is delivered from E3 to both E2 and E6, and the fifth pulse P5 is delivered from E4 to both E1 and E5. This pulse pattern can effectively target injectate dispersed between the electrode rows 817 using fewer total pulses than the pattern shown in FIG. 8B.

It should be appreciated that the example pulse patterns described above with reference to FIGS. 8A-8C represent non-limiting examples of pulse patterns that can be employed with the matrix array 815. It should also be appreciated that the foregoing pulse patterns can also be employed with the matrix array 715 shown in FIGS. 6A-6B. Furthermore, these pulse patterns can be adjusted as needed based on the particular factors involved.

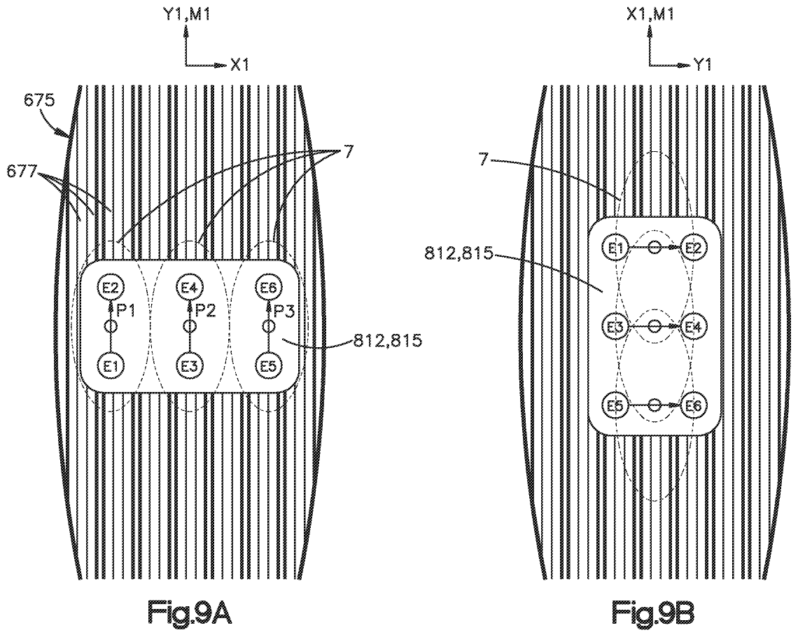

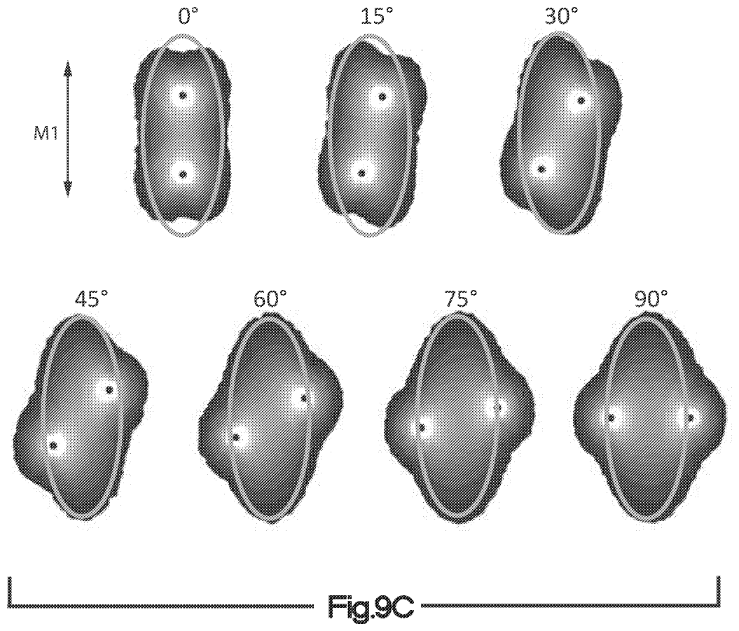

Referring now to FIG. 9A-9C, an additional advantage of the matrix array 815 described above with reference to FIGS. 7A-7B involves its particular effectiveness in tissues that influence fluid dispersion along specific directions. One such tissue is muscle tissue 675. As described above, intramuscular (IM) tissue tends to influence injected fluid 7 (e.g., the injectate) to disperse predominantly along the direction of muscle fiber extension M1. One particular advantage of the matrix array 815 is that its design allows favorable IM electroporation results regardless of its orientation relative to the direction of muscle fiber extension M1. In this manner, the matrix array 815 can be said to be more robust against mis-orientation in muscle.

As shown in FIG. 9A, the matrix array 815 can be inserted into muscle tissue 675 at an orientation whereby the electrode rows 817 align with the direction of muscle fiber extension M1. This orientation can be characterized as a “parallel” or “0-degree” orientation. In this orientation, each electrode row 817 and the associated injection channel 836 generally extends alongside and/or in-between the same muscle fibers 677. The three (3) fluid injections (utilizing the injection channels 836) disperse predominantly along the direction of muscle fiber extension M1, resulting generally in three side-by-side fluid dispersions 7. In this manner, each of electroporation pulses P1-P3 can effectively target the respective fluid dispersion 7 so that the high-magnitude portions of the electroporation fields co-localize with the respective fluid dispersions 7.

As shown in FIG. 9B, the matrix array 815 can alternatively be inserted into muscle tissue 675 at an orientation whereby the electrode rows 817 are oriented perpendicular to the direction of muscle fiber extension M1. This orientation can be characterized as a “perpendicular” or “90-degree” orientation. In this orientation, each electrode row 817 can traverse multiple muscle fibers 677. The three (3) fluid injections (utilizing the injection channels 836) disperse predominantly along the direction of muscle fiber extension M1, resulting generally in longitudinally overlapping fluid dispersions 7 having a maximum concentration between electrodes E3 and E4. In this manner, electroporation pulses P1-P3 can effectively target more muscle fibers and encompass more of the injected fluid than at the 0-degree orientation. Thus, a physician can employ the matrix array 815 at the 90-degree orientation to target more injectate with a more homogeneous electrical field, which can lead to transfecting more myocyte cells.

Referring now to FIG. 9C, each electrode pair (i.e., the electrodes in a single row 817) demonstrate strong co-localization of the electroporation field and the fluid dispersion regardless of the array orientation relative to the direction of muscle fiber extension M1. For example, at the 0-degree orientation, the high-magnitude portion of the electrical field aligns with the high-concentration portion of the fluid dispersion 7. One reason for this result is because the muscle fibers 677 demonstrate anisotropic electrical conductivity that is highest along the direction of muscle fiber extension M1. Thus, electrical impedance is minimized along direction M1. Additionally, muscle fibers provide a lower mechanical fluid impedance along the direction of muscle fiber extension M1, as discussed above. However, even when the orientation rotates toward higher angles, the injectate still disperses along the direction of muscle fiber extension M1 while the electrical field deforms (due to electrical conductivity being anisotropic and highest along the fiber axis) to somewhat match. Even at a 90-degree orientation, the electrical field is effectively “stretched” in direction M1, resulting in an electrical field that bulges out in the middle, where injectate is located. Thus, regardless of the array 815 orientation relative to muscle fibers, the array 815 beneficially co-localizes the electrical field with the injectate.

In other embodiments of the matrix array 815, the number of electrode rows 817 and/or columns 819 and/or the number of injection channel rows 840 and/or columns 842 of the matrix array 815 can be reduced or increased as needed based on various factors, such as the target treatment location, target tissue, and injection volume, by way of non-limiting examples. For example, the matrix array 815 can be increased to include one or more additional rows 817 and/or columns 819 of electrodes and/or one or more additional rows 840 and/or columns 842 of injection channels 836, such that the rows 840 of injection channels 836 are aligned with the rows 817 of electrodes 14. It should also be appreciated that the matrix array 815 can employ a combination of one or more injection channels 836 that are aligned with respective electrode rows 817 and one or more injection channels 836 that are offset from respective electrode rows 817 (including eccentrically offset or equidistantly offset).

It should be appreciated that the various parameters of the injection needles 20, 625 and associated array assemblies 212, 312, 412, 612, 712, 812 and/or electrode arrays 215, 315, 415, 615, 715, 815 described above are provided as exemplary features, such as for enhancing injection volumes within an expanded electroporation field and thereby enhancing electroporative transfection. These parameters can be adjusted as needed without departing from the scope of the present disclosure. For example, the illustrated electrode arrays and chimney arrays represent non-limiting examples of the array sizes and designs possible according to the embodiments herein. The electrode arrays and chimney arrays can be employed at virtually any array respective size (e.g., 15×15, 50×50, 100×100, and more than 100× more than 100). Moreover, the array assemblies disclosed herein can be adapted so that their electrode arrays and chimney arrays can approximate a shape of a patient's entire muscle or a portion thereof, including an entire length of a patient's muscle, including a long muscle, such as the sartorius muscle, by way of non-limiting examples. It should also be appreciated that the electrode arrays and/or chimney arrays can be arranged in various patterns, including staggered patterns, curved patterns, and irregular patterns, with can involve various spacing distances and/or non-uniform spacing distances.

It should be understood that when a numerical preposition (e.g., “first”, “second”, “third”) is used herein with reference to an element, component, dimension, or a feature thereof (e.g., “first” electrode, “second” electrode, “third” electrode), such numerical preposition is used to distinguish said element, component, dimension, and/or feature from another such element, component, dimension and/or feature, and is not to be limited to the specific numerical preposition used in that instance. For example, a “first” electrode, direction, or support member, by way of non-limiting examples, can also be referred to as a “second” electrode, direction, or support member in a different context without departing from the scope of the present disclosure, so long as said elements, components, dimensions and/or features remain properly distinguished in the context in which the numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An electrode array for use with an electroporation device, the electrode array comprising:
a support member having a top surface and a bottom surface, the support member defining a plurality of injection channels extending from the top surface to the bottom surface;
a plurality of needle electrodes coupled to the support member, such that distal ends of the plurality of needle electrodes extend to a needle depth below the bottom surface of the support member, wherein the plurality of needle electrodes are arranged in a matrix pattern having rows of the needle electrodes and columns of the needle electrodes disposed along the support member, and the plurality of injection channels are dispersed within the matrix pattern of the needle electrodes.

2. The electrode array of claim 1, wherein the support member includes a plurality of tube formations extending outwardly from the top surface, and the plurality of tube formations define extensions of the plurality of injection channels.

3. The electrode array of claim 2, wherein the plurality of tube formations define outer ends that are configured to mount with complimentary mounting formations of injection needles, such that distal ends of the injection needles extend to at least one second depth below the bottom surface when the mounting formations of the injection needles are seated with respect to the outer ends of the tube formations.

4. The electrode array of claim 3, wherein the second depth is less than the needle depth by at least 0.5 mm.

5. The electrode array of claim 3, wherein the second depth is less than the needle depth by at least 1.0 mm.

6. The electrode array of claim 3, wherein the second depth is less than the needle depth by at least 5 mm.

7. The electrode array of claim 1, wherein the matrix has four or more rows and two or more columns, and the support member defines at least two injection channels.

8. The electrode array of claim 1, wherein the matrix has five or more rows and two or more columns, and the support member defines at least two injection channels.

9. The electrode array of claim 1, wherein the matrix is a 3×2 matrix having six needle electrodes arranged in three rows and two columns, and the support member defines three injection channels.

10. The electrode array of claim 10, wherein the three injection channels are arranged in a single column equidistantly spaced between the two columns of needle electrodes.

11. The electrode array of claim 11, wherein the three injection channels are in-line with the three rows of needle electrodes.

12. The electrode array of claim 11, wherein the three injection channels are eccentrically offset from the three rows of needle electrodes at respective offset distances.

13. The electrode array of claim 1, wherein the support member has circuitry providing electrical communication to each of the plurality of needle electrodes individually, such that select subsets of needle electrodes are configured to deliver electroporation pulses.

14. An electroporation device for causing in vivo reversible electroporation in cells of tissue, comprising:
an electrode array that includes:
a support member having a top surface and a bottom surface, the support member defining a plurality of injection channels extending from the top surface to the bottom surface;
a plurality of needle electrodes coupled to the support member, such that distal ends of the plurality of needle electrodes extend to a needle depth below the bottom surface of the support member, wherein the plurality of needle electrodes are arranged in a matrix pattern having rows of the needle electrodes and columns of the needle electrodes disposed along the support member, and the plurality of injection channels are dispersed within the matrix pattern of the needle electrodes; and
a plurality of injection needles configured to extend through at least some of the plurality of injection channels and into the tissue.

15. The electroporation device of claim 14, further comprising an applicator having a handle and a mounting portion connected to the handle, wherein the electrode array is attachable to the mounting formation, and the plurality of needle electrodes are in communication with circuitry of the applicator for controlling delivery of one or more electroporative pulses to the plurality of needle electrodes when the electrode array is attached to the mounting formation.

16. The electroporation device of claim 14, wherein the support member includes a plurality of tube formations extending outwardly from the top surface, and the plurality of tube formations define extensions of the plurality of injection channels.

17. The electroporation device of claim 16, wherein the plurality of tube formations define outer ends that are configured to mount with complimentary mounting formations of the injection needles, such that distal ends of the injection needles extend to at least one second depth below the bottom surface when the mounting formations of the injection needles are seated with respect to the outer ends of the tube formations.

18. The electroporation device of claim 17, wherein the second depth is less than the first depth by at least 0.5 mm.

19. The electroporation device of claim 17, wherein the second depth is less than the needle depth by at least 1.0 mm.

20. The electroporation device of claim 17, wherein the second depth is less than the needle depth by at least 5 mm.

21. The electroporation device of claim 14, wherein the matrix has four or more rows and two or more columns, and the support member defines at least two injection channels.

22. The electroporation device of claim 1, wherein the support member has circuitry providing electrical communication to each of the plurality of needle electrodes individually, such that select subsets of needle electrodes are configured to deliver electroporation pulses.

23. An electroporation system for causing in vivo reversible electroporation in cells of tissue, comprising:
an electrode array that includes:
a support member having a top surface and a bottom surface, the support member defining a plurality of channels extending from the top surface to the bottom surface;
a plurality of needle electrodes coupled to the support member and extending through the plurality of channels, such that distal ends of the plurality of needle electrodes extend to a needle depth below the bottom surface of the support member, wherein the plurality of needle electrodes are arranged in a matrix pattern having rows of the needle electrodes and columns of the needle electrodes disposed along the support member, wherein at least some of the plurality of needle electrodes are dual-purpose injection needle electrodes configured to inject an agent into the tissue and to deliver one or more electroporation pulses to the tissue for causing the reversible electroporation in the cells of the tissue.

24. The electroporation system of claim 23, further comprising an applicator having a handle and a mounting portion connected to the handle, wherein the electrode array is attachable to the mounting formation, and the plurality of needle electrodes are in communication with circuitry of the applicator for controlling delivery of one or more electroporative pulses to the plurality of needle electrodes.

25. The electroporation system of claim 24, further comprising tubing connected to and in fluid communication with the dual-purpose injection needle electrodes, wherein the tubing is configured for delivering injectate from a reservoir assembly to the dual-purpose injection needle electrodes.

26. The electroporation system of claim 25, wherein all of the plurality of needle electrodes are dual-purpose injection needle electrodes.
